(19) United States
(12) Patent Application Publication
(10) Pub. No.: US 2017/0065287 A1
(43) Pub. Date: Mar. 9, 2017

(54) Infrared Endoscopic Probe

(71) Applicants: Octavio Cesar Silva, Melbourne, FL (US); Fernando Emilio Silva, Westminster, CA (US)

(72) Inventors: Octavio Cesar Silva, Melbourne, FL (US); Fernando Emilio Silva, Westminster, CA (US)

(21) Appl. No.: 15/333,172

(22) Filed: Oct. 24, 2016

Related U.S. Application Data
(63) Continuation-in-part of application No. 13/872,122, filed on Apr. 28, 2013, now abandoned.

(51) Int. Cl. A61B 17/16; A61B 1/00; A61B 1/12; A61B 1/07

(52) U.S. Cl. CPC A61B 17/1671; A61B 1/07; A61B 1/00195; A61B 1/00117; A61B 1/00087; A61B 1/12; A61B 17/1615

(57) ABSTRACT

The Infrared Endoscopic Probe represents a new instrument to bore pilot holes in vertebra pedicles while imaging the operation in the infrared spectrum with an integrated fiber optics endoscope. The pilot holes are bored to provide entry points for pedicle screws that serve as anchor points for spine stabilizing rods to treat several spine conditions. The device consists of a metal body that terminates in a tapered incision tip, an endoscope that runs inside said metal body, a handle to drive the device into pedicle boney tissue and a fiber optics harness that enters the device handle and is used to connect to imaging, illumination, irrigation and suction devices to enable the endoscopic functions of the device. The fiber optics harness connects to an imaging camera that provides an electrical signal to a monitor to view the operation in real time. A method is described to accomplish this procedure.

### CROSS REFERENCE TO RELATED APPLICATIONS

This is a non-provisional patent application submitted as a continuation-in-part application corresponding to non-provisional patent application Ser. No. 13/872,122, Infrared LOB Probe submitted on Apr. 28, 2013.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not Applicable.

### DESCRIPTION OF DRAWINGS

FIG. 1 shows embodiment 1 of the overall architecture of the device. The device provides an external interface to a solid state imaging camera that provides an electrical signal to a monitor to view video. It also provides interfaces to an infrared illumination source such as a Light Emitting Diode (LED) and to an irrigation/suction device.

FIG. 2 shows embodiment 1 of the optical distal end layout. The diagram is a cross section that depicts one objective lens system and two illumination lens systems as well as a conduit for irrigation or air suction.

FIG. 3 shows embodiment 2 of the optical distal end layout. The diagram is a cross section that depicts one objective lens system and two illumination lens systems. It also shows an irrigation conduit above the objective lens system and two suction conduits, one on each side of the objective lens system.

FIG. 4 shows embodiment 3 of the optical distal end layout. The diagram is a cross section that depicts one objective lens system and two illumination lens systems. The illumination lens systems are on one side while the objective lens system is on the other side and separated from them by the suction opening. The Irrigation opening is above the objective lens system.

FIG. 5 shows embodiment 4 of the optical distal end layout. The diagram is a cross section that depicts one objective lens system and two illumination lens systems. The illumination lens systems are on one side while the objective lens system is on the other side. A suction and irrigation conduits are between the illumination and imaging fiber cores, the suction opening being next to the illumination fiber cores while the irrigation opening being next to the imaging fiber core.

FIG. 6 shows embodiment 5 of the optical distal end layout. The diagram is a cross section that depicts one objective lens system and two illumination lens systems.

FIG. 7 shows embodiment 2 of the architecture of the IR Endoscopic Probe. The device provides an external interface to a solid state imaging camera, which provides an electrical signal to a monitor to view video. It also provides interfaces to an infrared illumination source, to an irrigation device and to a suction device. The irrigation and suction conduits are separate.

FIG. 8 shows embodiment 3 of the IR Endoscopic Probe architecture. The device provides an external interface to a solid state imaging camera, which provides an electrical signal to a monitor to view images. It also provides an interface to an infrared illumination source.

FIG. 9 shows a cross section of the objective lens system of embodiment 1 of the optical distal end. The cross section is on a plane perpendicular to the IR Endoscopic Probe body.

FIG. 10 shows a cross section of one of the illumination lens systems of embodiment 1 of the optical distal end. The cross section is on a plane perpendicular to the IR Endoscopic Probe body.

FIG. 11 shows a cross section of embodiment 1 of the optical distal end. The cross section is on a plane horizontal to the IR Endoscopic Probe body and shows the objective lens system and the illumination lens systems.

FIG. 12 shows a cross section of embodiment 4 of the optical distal end. The cross section is on a plane horizontal to the IR Endoscopic Probe body and shows the objective lens system and the illumination lens systems plus the irrigation and suction conduits.

FIG. 13 shows a cross section of the slant design of embodiment 1 of the optical distal end. The cross section is on a plane perpendicular to the IR Endoscopic Probe through the objective lens system and irrigation conduit.

FIG. 14a shows a cross section of the slant design of embodiment 1 of the optical distal end. The cross section is on a plane perpendicular to the IR Endoscopic Probe through one of the illumination lens systems.

FIG. 14b shows a cross section of the flush design of embodiment 1 of the optical distal end. The cross section is on a plane perpendicular to the IR Endoscopic Probe through one of the illumination lens systems.

FIG. 15a shows the front of the tip of the IR Endoscopic Probe with the layout of the objective lens system and the illumination lens systems.

FIG. 15b shows the front of the tip of the IR Endoscopic Probe with the layout of the objective lens system and the illumination lens systems.

FIG. 16 shows the coupling optics of the imaging fiber bundle. The figure shows the coupling to a solid state imaging camera optical assembly.

FIG. 17 shows the coupling optics of the illumination fiber bundles. The figure shows the coupling to an infrared illumination source.

FIG. 18 shows the coupling to the irrigation and suction devices through the same connector.

FIG. 19 shows the coupling to the suction device.

FIG. 20 shows the coupling to the irrigation device.

FIG. 21 shows a sample tip of a IR Endoscopic Probe without the optical distal end.

FIG. 22 shows a generalized flow chart of the process to create pilot holes in vertebrae.

### SUMMARY OF THE INVENTION

The IR Endoscopic Probe represents a new way to safely place pedicle screws while imaging the operation with the aid of fiber optics technology. The operation is performed to create holes in the vertebrae to provide an entry point for screws for various spinal conditions. Currently, using a free-hand technique, fluoroscopy and/or image guidance, a probe is used to blindly create a pilot hole. The probe path is radiographically imaged to ensure that the probe follows a proper path. The probe is re-directed and the pilot hole is completed. The pilot hole is tapped, blindly, and a screw inserted. The IR Endoscopic Probe provides real time imaging and continuous monitoring of the pilot hole creation into bone, allowing re-direction of the probe as necessary to avoid vital vascular and neural structures. This not only leads to safer and more accurate screw placement, but optimized screw length and diameter, as imaging, fluoroscopy in particular, can be misleading. The disposable design of the IR Endoscopic Probe also ensures sharpness and optimal optics in every use. This method builds on a classic method of spinal instrumentation, and regardless of spinal deformity, allows the surgeon for safe and accurate spinal instrumentation avoiding the inherent dangers of radiation and use of very expensive guidance systems.

### DETAILED DESCRIPTION OF THE INVENTION

Non-provisional patent application No. 13872122 is described herein with amendments, namely four new layouts of the fiber optics cores in the Infrared Endoscopic Probe (IR Endoscopic Probe), an additional introduction of separate suction and irrigation conduits, a design without irrigation and suction conduits, and generalized placement of the optical distal end in the device incision tapered tip. In addition, representative objective and illumination lens systems for the optical distal end are described. Notwithstanding, the same geometry of the device is described as well as the same fiber optics harness with the single suction/irrigation conduit.

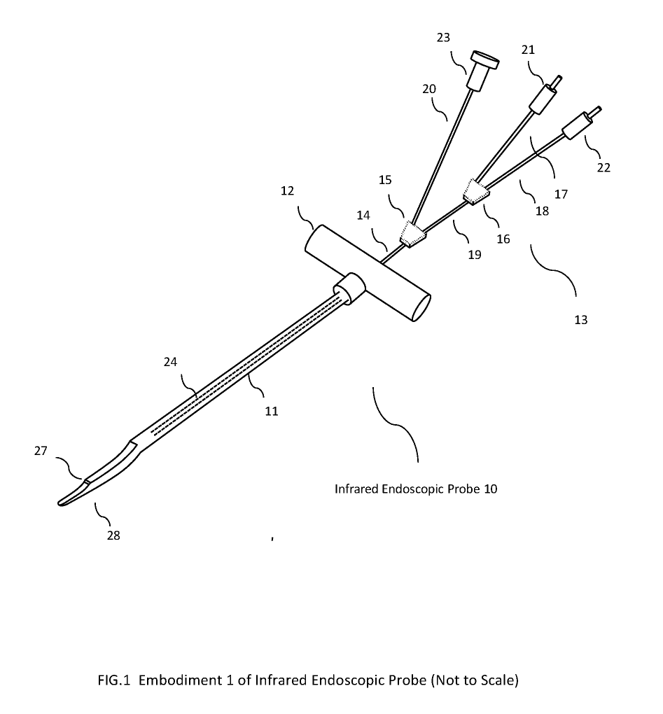

As shown in FIG. 1, embodiment 1 of the IR Endoscopic Probe 10 design consists of a metal body 11, the integrated endoscope 24 that runs inside the length of the metal body 11, a handle 12 to drive the IR Endoscopic Probe and a fiber optics harness 13 that enters the handle 12. The device is also comprised of an imaging device and an illumination device. The probe terminates in a tapered tip 28 used to make incisions in vertebrae. The endoscope optical distal end is located at a distance d1 from the tapered tip front whose length varies from 2.5 cm to 5 cm.

The IR Endoscopic Probe allows surgeons to precisely create pilot holes in pedicle bones, which are created to place pedicle screws to anchor spine stabilizing rods. This method is unique since by choosing the proper infrared light wavelength tissue becomes transparent allowing a surgeon to select the proper path for the boring of the pilot holes. Light in the visible spectrum, for example at blue or green wavelengths or white light for that matter, can only be used to view bone structure next to the endoscope objective lens since light in the visible spectrum cannot penetrate bone tissue virtually. However, penetration of bone tissue can be achieved at infrared wavelengths notably in the near infrared spectrum region around 950 nm where penetrations can be of the order of 3 to 4 cm. Other longer infrared wavelengths allow similar penetrations where windows of low attenuation exist, for example at 1600 nm. Since the diameter of the pedicle is less than 2 cm, inspection of nerve and vascular structures outside of the pedicle bone is possible when the infrared light is intense. Therefore, not only can the surgeon inspect the bone structure immediately next to the imaging less but also see beyond as bone tissue becomes transparent and determine whether the probe is following a path toward outer nerve and vascular structures located next to the outer surface of the pedicle bone. This operation can be viewed in its entirety in real-time in a video monitor at the selected infrared wavelength such that pilot holes can be created solely in bone tissue while avoiding vital outer vascular and nerve structures. The device is designed to create such pilot holes in that the tapered tip is shaped in such a way to perforate bone tissue in the same fashion as a pick.

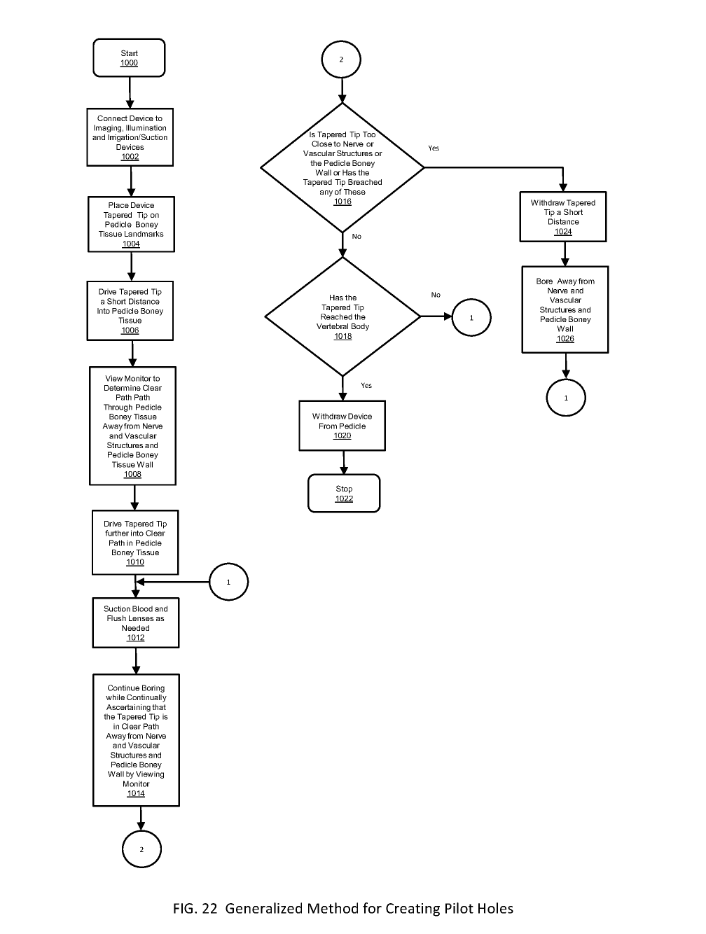

FIG. 22 describes a generalized method to create pilot holes in vertebrae using the IR Endoscopic Probe. The IR Endoscopic Probe is connected to infrared imaging, infrared illumination and irrigation/suction devices to enable its endoscopic functions at the selected infrared light wavelength 1002. The tapered tip 28 is placed on the selected vertebra's pedicle at predetermined boney landmarks 1004. The tapered tip 28 is then driven a short distance into the pedicle boney tissue by manipulating the surgical device with its handle 1006 while determining in the monitor a clear path through pedicle boney tissue away from nerve or vascular structures or the pedicle boney wall 1008. Next, the tapered tip 28 is further driven into said clear path in the pedicle boney tissue 1010. Blood is suctioned as needed while the lens systems are flushed as needed in case of obstructions 1012. Boring is continued while continually ascertaining that the tapered tip 28 is in said clear path through pedicle boney tissue away from nerve and vascular structures and the pedicle boney wall by viewing the operation in the monitor 1014. A determination is made whether the tapered tip 28 is too close to nerve or vascular structures or the pedicle boney wall or whether the tapered tip has breached any of these 1016. If not, another determination is made whether tapered tip has reached the vertebral body 1018. If so, the device is withdrawn 1020 as the pilot hole has been created and the process is ended 1022. If in determination 1016 the tapered tip is too close to the nerve or vascular structures or the pedicle boney wall or if the tapered tip has breached any of these, the tapered tip 28 is withdrawn a little 1024 while continuing boring away from nerve and vascular structures and the pedicle boney wall 1026 as the process continues to process step 1012. If in determination 1018 the tapered tip has not reached the vertebral body, the process continues to process step 1012. The length of the pilot hole is determined from the markings imprinted on the vertebral pick device. Step 1012 is omitted in embodiment 3 of the IR Endoscopic Probe since this embodiment lacks irrigation and suction conduits.

Infrared light illumination from an LED source or similar source is carried by two fiber cores which are embedded in the channels in the IR Endoscopic Probe body 11, 31, and 61 in FIGS. 1, 7 and 8, respectively. At the optical distal end output, these two fiber cores illuminate the region where the IR Endoscopic Probe is to make the incision in the vertebra. The other fiber core carries the imaging of the incision operation in the infrared light spectrum. The imaging is carried to an external optical assembly and solid state imaging camera, connected to the imaging fiber bundle 17. In addition to the fiber cores, the irrigation/suction conduit runs along the length of the IR Endoscopic Probe body. The endoscope resides in the metal body only as defined herein and consists of an imaging system and two illumination systems. The endoscope extensions to the optical fibers external to the metal body form part of the fiber optics harness. The imaging system consists of an imaging fiber bundle and an objective lens system, which is comprised of three lenses plus an optical window as exemplified herein. Each illumination system consists of an illumination fiber bundle plus an illumination lens system, which is comprised of a single lens plus an optical window as exemplified herein. The fiber cores are the endoscope fiber bundles laid out in the channels that run through the elongated metal body.

The Infrared Endoscopic Probe structure is designed with a narrow and long tapered tip, curved to follow the curvature of the pedicle bone structure as the pedicle bone ends in the vertebral bone. The width of the tapered tip is typically 4 mm at the proximal end and gradually decreases to a pointy, sharp distal end. The height of the tapered tip is typically 5 mm at the proximal end and gradually curves to the pointy, sharp distal end. The tapered tip has to be rigid, stiff and essentially solid with a sharp distal end to perforate bone tissue sharply and smoothly. The wall of the pilot hole has to be smooth so that the pedicle screw remains rigidly fixed when it is inserted after the pilot hole is created. The length of the tapered tip varies between approximately 2.5 cm to 5 cm depending on the length of the pedicle bone which varies depending on the region of the spine.

Therefore, the optical distal end of the endoscope has to be sturdy and rigid to withstand the shock of the boring operation of the pilot hole and the optical windows have to be hard. In one embodiment, the optical distal end can be integrated on top of the tapered tip at a predetermined distance (4 mm-15 mm) from the tapered tip distal end. This location on top of the tapered tip ascertains the direction that the tapered tip distal end is following (the section of the tapered tip that the objective lens system “sees”) even before it perforates any tissue and is necessary in cases when the upper hemisphere is sufficient for imaging and illumination. This is necessary only when imaging of some sections of the internal pedicle bone tissue is required. The small step is also necessary in terms of manufacturing a more cost effective IR Endoscopic Probe than the other options. The alternatives are the slant step and the flush window design which would allow for a smoother insertion into the pedicle bone. The slant step would cause essentially no distortion in imaging while the flush window design would cause some distortion but not significantly.

In another embodiment, the optical distal end is located in front of the tapered tip distal end which is necessary when all the surroundings in front have to be imaged as the pilot hole is perforated in cases where the pedicle bone is narrow since the objective lens system field of view is not obstructed and when more immediate detection of the vertebra bone at the end is required. However, this design could be more difficult to manufacture since the optical windows have to be geometrically flush in the front.

The handle structure has the shape of a T to exert more torque to the elongated metal body. Horizontal section of the T-shaped handle has a length of more than 8 cm. The elongated metal body is more than 10 cm in length also to exert more torque on the pedicle bone tissue. Connections to the external imaging device and illumination imaging device is more practical with a flexible fiber optics harness. A flexible harness is difficult to break while the flexible entry point in the handle allows for more unobstructed manipulation of the IR Endoscopic Probe with its handle.

Embodiment 2 of the IR Endoscopic Probe is the same as embodiment 1 except that the fiber optics harness 33 has two separate irrigation and suction conduits instead of having a single irrigation/suction conduit. Also, embodiment 2 has separate irrigation and suction conduits in the metal body 31.

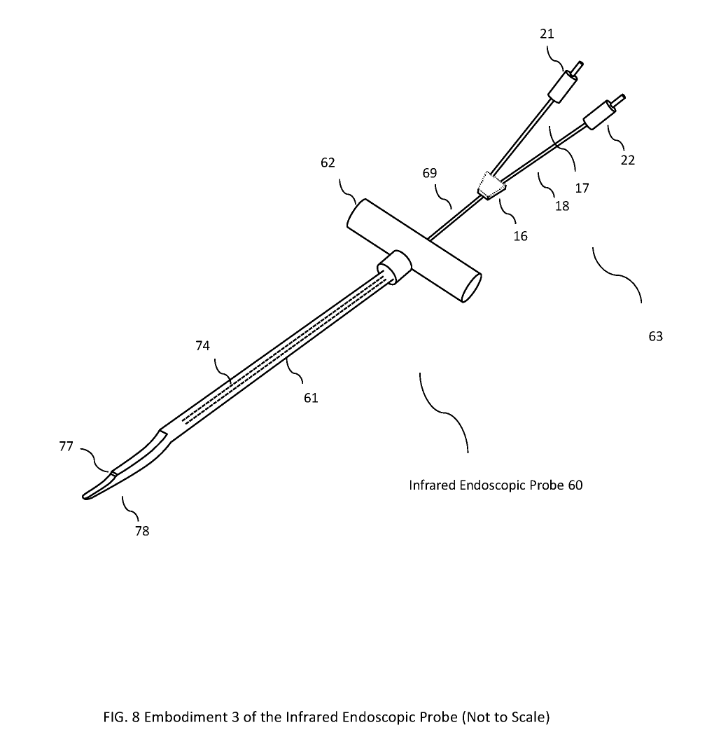

Embodiment 3 of the IR Endoscopic Probe device, shown in FIG. 8, is the same as embodiment 1 except that the fiber optics harness 63 has no irrigation/suction conduit and the metal body 61 just has channels for one imaging fiber core and two illumination fiber cores.

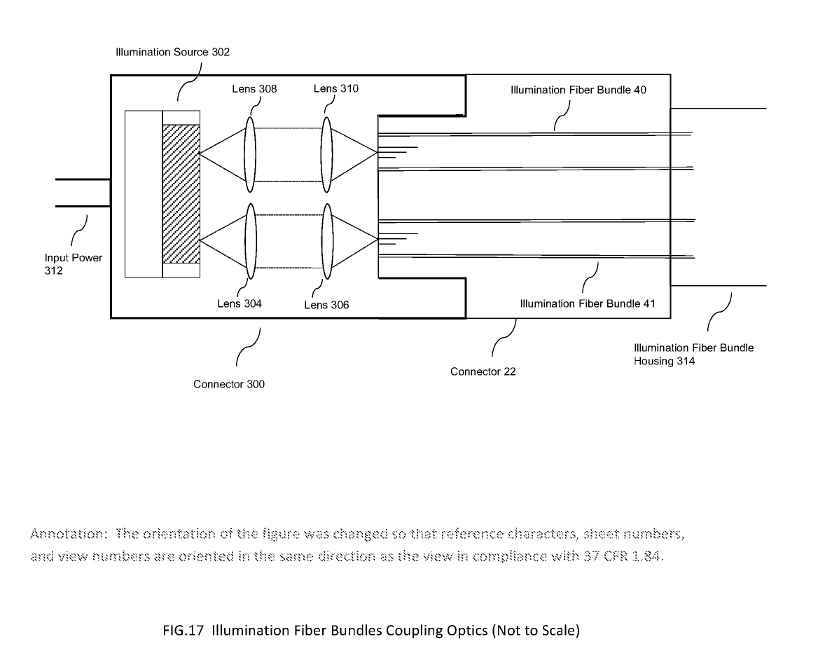

In embodiment 1 of the fiber optics harness 13, the IR Endoscopic Probe 10 provides three connectors 21, 22 and 23. Connector 21 couples with the imaging camera optics connector (not shown) while connector 22 couples with the illumination source optics connector (not shown). The Illumination fiber assembly 18 encases two illumination fiber bundles 40 and 41 as shown in FIG. 17. The imaging fiber bundle 17 which is encased singly and illumination fiber assembly 18 merge at the fiber optics merging point 16, which is a plastic molded junction from where the fiber bundles emerge into a single housing 19 which encases them. Connector 23 couples with the irrigation or suction device and is the terminating point for the irrigation/suction conduit 20, an encased plastic tube. Conduit 20 merges with the fiber optics assembly 19 at junction 15 from which a single integrated housing 14 emerges that encases the imaging fiber bundle, the illumination fiber bundles and the irrigation/suction conduit. The integrated housing 14 enters the IR Endoscopic Probe handle 12 where the imaging fiber bundle continues to the imaging fiber core in the IR Endoscopic Probe metal body 11, each illumination fiber bundle continues to its respective illumination fiber core and the irrigation/suction conduit continues to the irrigation/suction channel. Continuation, in this case, refers to transitioning or extending to a different part of the device since each element, for example, the imaging fiber core and the imaging fiber bundle are the same. That is, they are the same set of fibers placed in a different location of the device. They are named differently to characterize this location since each location houses them differently.

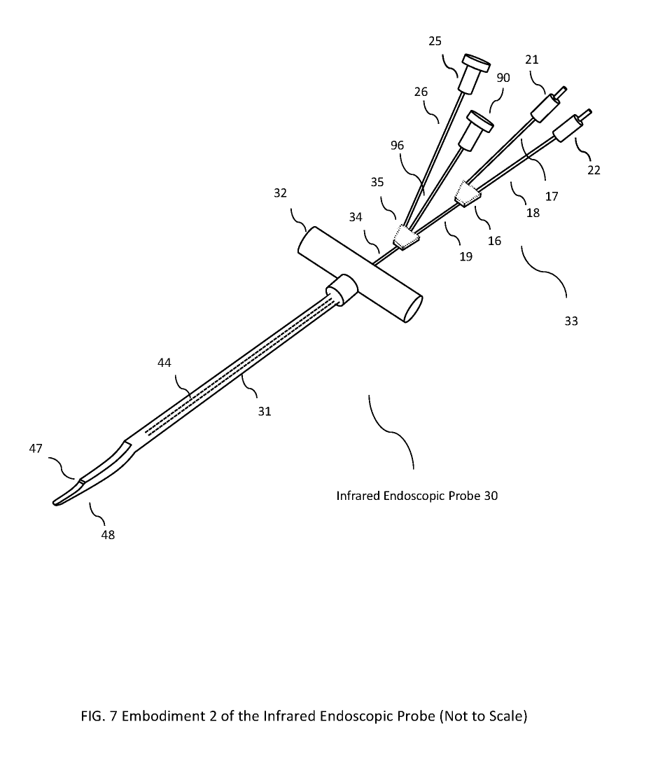

In embodiment 2 of the fiber optics harness 33, shown in FIG. 7, the IR Endoscopic Probe 10 provides four connectors 21, 22, 90 and 25. Connector 21 couples with the imaging camera optics connector (not shown) while connector 22 couples with the illumination source optics connector (not shown). Illumination fiber assembly 18 encases two illumination fiber bundles 40 and 41 as shown in FIG. 17. The imaging fiber bundle 17 which is encased singly and illumination fiber assembly 18 merge at the fiber optics merging point 16, which is a plastic molded junction from where the fiber bundles emerge into a single housing 19 which encases them. Connector 90 couples with the irrigation device and is the terminating point for the irrigation conduit 96, an encased plastic tube, while connector 25 couples with the suction device and is the terminating point for the suction conduit 26, an encased plastic tube. Conduits 20 and 96 merge with the fiber optics assembly 19 at junction 35 from where a single integrated housing 34 emerges that encases the imaging fiber bundle, the illumination fiber bundles and the irrigation/suction conduit. The integrate housing 34 enters the IR Endoscopic Probe handle 32 where the imaging fiber bundle continues to the imaging fiber core in the IR Endoscopic Probe metal body 31, each illumination fiber bundle continues to its respective illumination fiber core, the irrigation conduit continues to the irrigation conduit and the suction conduit continues to the suction conduit. Continuation, in this case, refers to transitioning or extending to a different part of the device since each element, for example, the imaging fiber core and the imaging fiber bundle are the same. That is, they are the same set of fibers placed in a different location of the device. They are named differently to characterize this location since each location houses them differently.

Embodiment 3 of the fiber optics harness 63, FIG. 8, is the same as embodiment 1 of the fiber optics harness except that there is no irrigation/suction conduit. As a result, the fiber optics integrated housing 69 that emerges from junction 16 encases the imaging fiber bundle and the illumination fiber bundles, enters the handle 62 and the design is devoid of junction 15 and conduit 20.

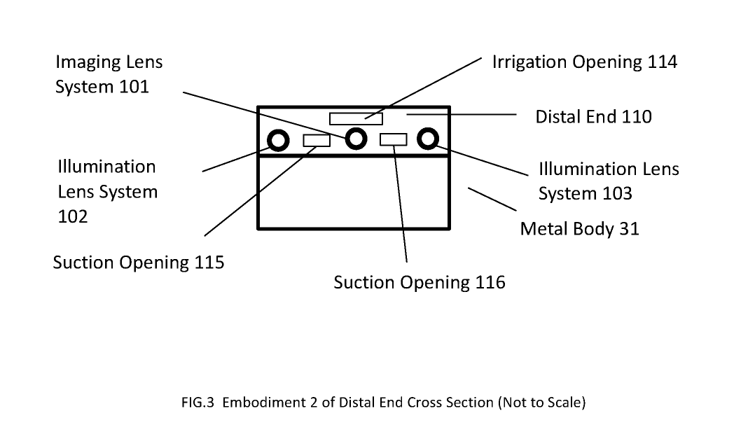

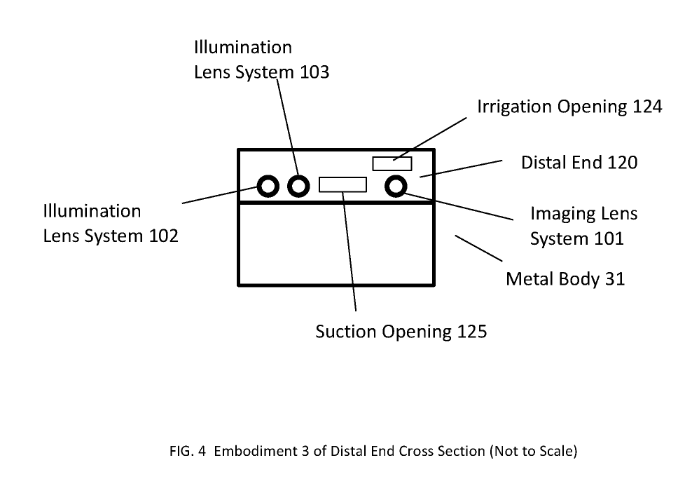

A similar design of the fiber optics harness is described in U.S. Pat. No. 4,576,145 Koichi Tsuno, et al, Mar. 18, 1986. The harness described by Tsuno provides connectors to imaging, illumination and irrigation devices. This is shown in FIG. 3 (items 7, 11, 17, 25). Another similar design is described in U.S. Pat. No. 5,127,393 by McFarlin, et al, Jul. 7, 1992. This is shown in FIG. 4 (items 58, 62, 66). Furhermore, a similar design is described in U.S. Pat. No. 5,263,928 by Trauthen et al (Nov. 23, 1993) in FIG. 1 (items 60, 62, 66, 64, 58, 24, 56, 52, 20). These patents also describe the design of thin endoscopes. These patents have expired and therefore use of those designs could be incorporated in the designs described in the present patent application.

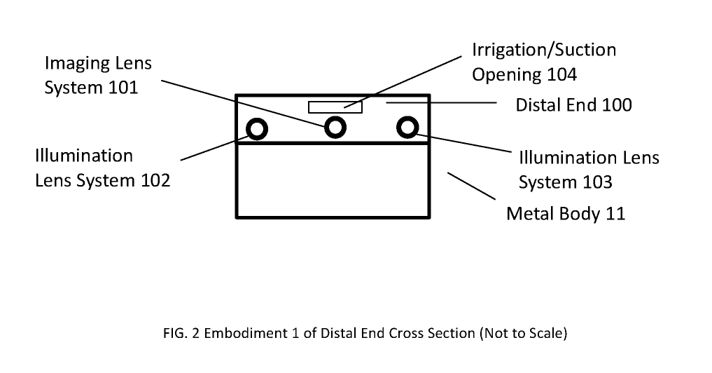

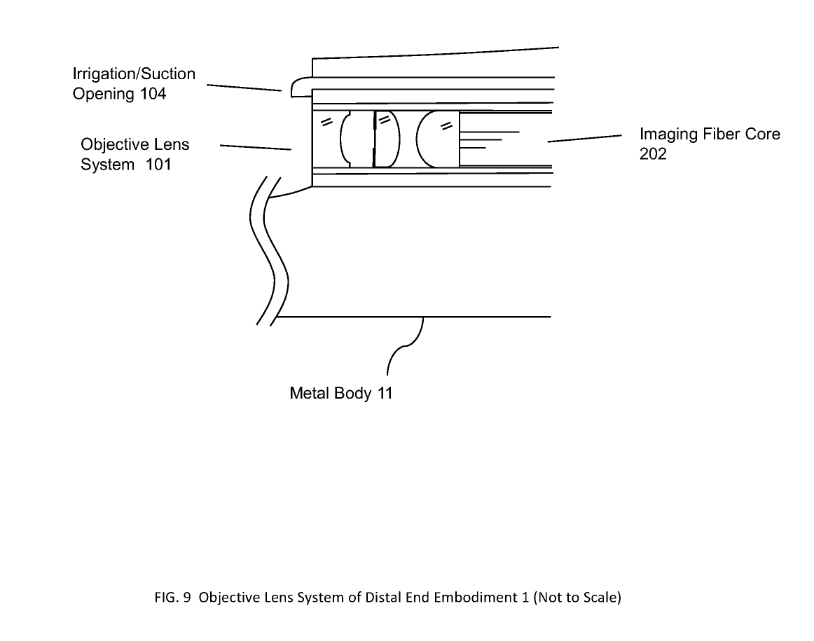

The endoscope is embedded inside of the metal probe and contains three fiber cores. A cross section of embodiment 1 of the optical distal end 100 is shown in FIG. 2. The optical distal end creates a small step, typically less than 2 mm, on top of the tapered tip. The objective lens system 101 is encased in the imaging channel and is located in the middle of the probe. The illumination lenses 102 and 103 are located on both sides of the optical distal end. An irrigation and suction channel 104 is located on top of the objective lens system to clear blood and other debris and maintain an unobstructed field of view. This opening terminates in a protruding bend to protect it from impact as shown in FIG. 9. The same channel is used to suction excess blood, so an external device provides the switch to change between irrigation and suction modes. The endoscope is part of the probe metal design for which channels are created along the metal body to accommodate the fiber cores, the optical distal end lenses and the irrigation and suction conduits.

Referring to the distal end design in FIG. 2, a cross section of the optical distal end on a plane perpendicular to the IR Endoscopic Probe body 11 is shown in FIG. 9. The plane slices the middle of the optical distal end and shows the arrangement of the objective lens system 101 and imaging fiber core 202 as well as the irrigation/suction channel 104 on top of the objective lens system. The irrigation nozzle terminates in a bend to protect the opening from impact and to direct the water flow towards the objective lens system. Although the figure shows the patented lenses described in paragraph [00054], other lens designs could be used.

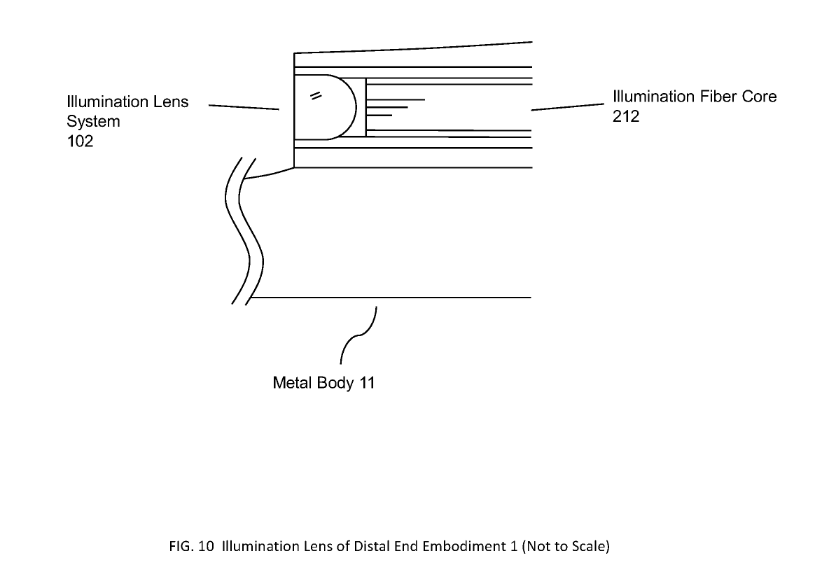

In addition, a cross section of the optical distal end on a plane perpendicular to the IR Endoscopic Probe body 11 is shown in FIG. 10. The plane slices one side of the optical distal end through the illumination lens 102, which is shown along with its respective illumination fiber core 212. Although the figure shows the patented lenses described in paragraph [00055], other lens designs could be used.

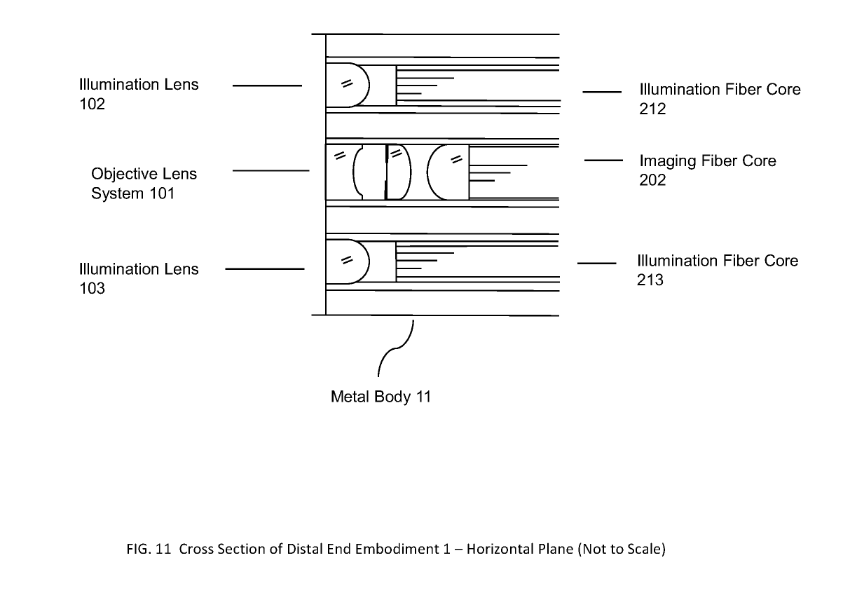

Another view of the optical distal end design in FIG. 2 is shown on a plane horizontal to the IR Endoscopic Probe body 11 in FIG. 11. The figure shows the arrangement of the objective lens system 101 and imaging fiber core 202 as well as the illumination lenses 102/103 and their respective fiber cores 212/213. Although the figure shows the patented lenses described in paragraphs [00054] and [00055], other lens designs could be used. The irrigation and suction opening is above the horizontal plane on top of the objective lens system and is not shown.

In embodiment 2 of the distal end, a cross section of the optical distal end 110 is shown in FIG. 3. The optical distal end creates a small step, typically less than 2 mm, on top of the tapered tip. The objective lens system 101 is encased in the imaging channel and is located in the middle of the probe. Next to the objective lens system are two suction openings 115 and 116 that bifurcate from the main suction channel that runs along of the probe metal body 31. Each opening may be located within 1.5 mm from the objective lens system. These openings terminate in a protruding bend to protect them from impact in the same manner as that of the irrigation opening. The illumination lenses 102 and 103 are located on both sides of the optical distal end. An irrigation channel 114 is located on top of the objective lens system to clear blood and other debris and maintain an unobstructed field of view. This opening terminates in a protruding bend to direct liquid flow and to protect it from impact. The endoscope is part of the probe metal design for which channels are created along the metal body to accommodate the fiber cores, the optical distal end lenses and the irrigation and suction conduits.

In embodiment 3 of the distal end, a cross section of the optical distal end 120 is shown in FIG. 4. The optical distal end creates a small step, typically less than 2 mm, on top of the tapered tip. The objective lens system 101 is encased in the imaging channel and is located on the right side. The illumination lenses 102 and 103 are located on the left side of the optical distal end. An irrigation channel 124 is located on top of the objective lens system to clear blood and other debris and maintain an unobstructed field of view. This opening terminates in a protruding bend to direct liquid flow and to protect it from impact in a similar manner as shown in FIG. 9. Next to the objective lens system is the suction opening 125 of the corresponding suction conduit that runs along the metal body of the probe. This opening terminates in a protruding bend to protect it from impact in the same manner as the irrigation opening. The endoscope is part of the probe metal design for which channels are created along the metal body to accommodate the fiber cores, the optical distal end lenses and the irrigation and suction conduits.

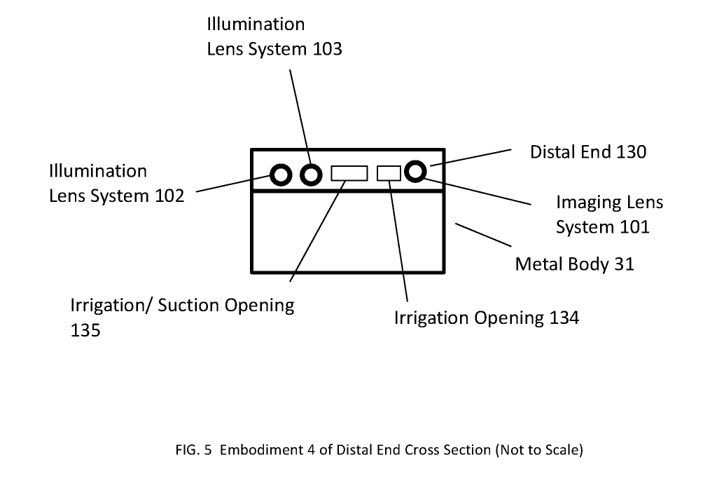

In embodiment 4 of the distal end, a cross section of the optical distal end 130 is shown in FIG. 5. The optical distal end creates a small step, typically less than 2 mm, on top of the tapered tip. The objective lens system 101 is encased in the imaging channel and is located on the right side of the optical distal end. The illumination lenses 102 and 103 are located on the left side of the optical distal end. An irrigation channel 134 is located next to the objective lens system to clear blood and other debris and maintain an unobstructed field of view. This opening terminates in a protruding bend that points sideways toward the objective lens system to direct liquid flow and to protect it from impact. Next to the irrigation conduit is the suction opening 135 of the corresponding suction conduit that runs along the metal body of the probe. The endoscope is part of the probe metal design for which channels are created along the metal body to accommodate the fiber cores, the optical distal end lenses and the irrigation and suction conduits.

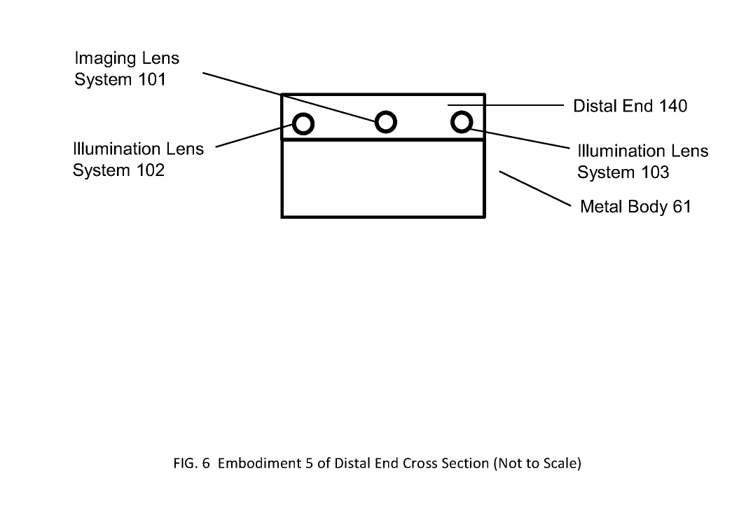

A cross section of embodiment 5 of the distal end 100 is shown in FIG. 6 and is the representation of embodiment 3 of the IR Endoscopic Probe design and embodiment 3 of the fiber optics harness. The optical distal end creates a small step, typically less than 2 mm, on top of the tapered tip. The objective lens system 101 is encased in the imaging channel and is located in the middle of the probe. The illumination lenses 102 and 103 are located on both sides of the optical distal end. The endoscope is part of the probe metal design for which channels are created along the metal body to accommodate the fiber cores, the optical distal end lenses and the irrigation and suction conduits.

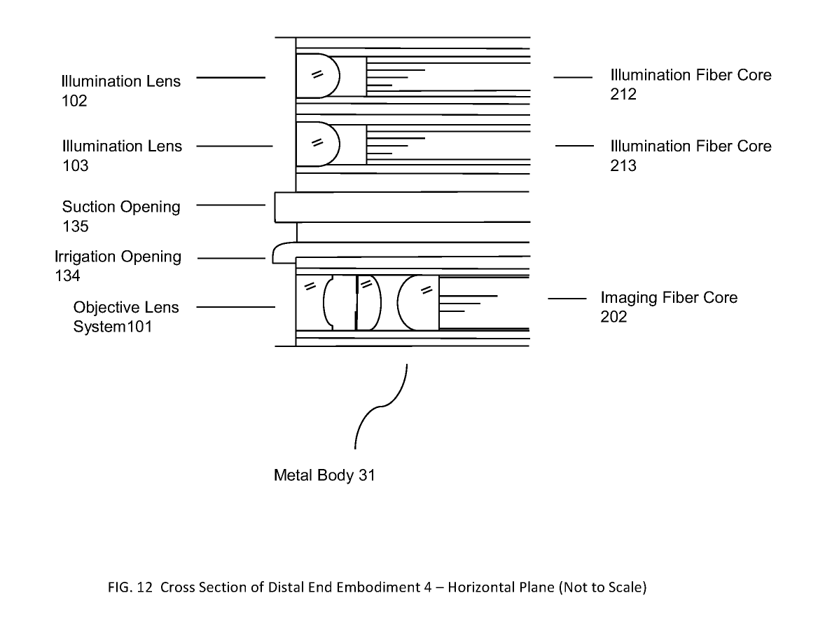

Referring to the distal end design in FIG. 5, FIG. 12 shows a cross section of the optical distal end on a plane horizontal to the IR Endoscopic Probe body 31. The figure shows the arrangement of the objective lens system 101 and imaging fiber core 202 as well as the illumination lenses 102/103 and their respective fiber cores 212/213. Although the figure shows the patented lenses described in paragraphs [00054] and [00055], other lens designs could be used. The figure also shows the arrangement of the Irrigation opening where the nozzle bend points towards the objective lens system. In addition, the figure depicts the suction opening where the nozzle bend points down.

Embodiments 2, 3 and 4 of the distal end are different instantiations of embodiment 2 of the IR Endoscopic Probe in that the fiber cores, the irrigation and suction conduits that run along the length of the metal body 31 are placed differently inside the body.

Objective and Illumination lens designs could be implemented by several of the patented designs published in the literature. The patents described herein are expired and can be incorporated in the designs described in the present patent applications. Patent U.S. Pat. No. 4,984,878 FIG. 11 shows a side view of an objective lens system design. This lens system is composed of three lens elements wherein the first lens on the object side is a plano-concave negative lens with the plane surface on the object side. The second lens is a plano-convex lens having the plane surface on the object side and finally a third plano-convex lens having the plane side on the image side and contiguous to the FO bundle that carries the imaging. The trade-off in this case is to miniaturize the lens diameter as much as possible while keeping a wide field of view.

A candidate illumination lens design is described in U.S. Pat. No. 7,585,274 FIG. 28 and consists of a single plano-convex lens element with the plane side on the object side. A FO bundle located at a distance d2 from the convex side carries light from a light source and transmits it through the lens. This lens design is suitable for miniaturization while keeping a wide field of view.

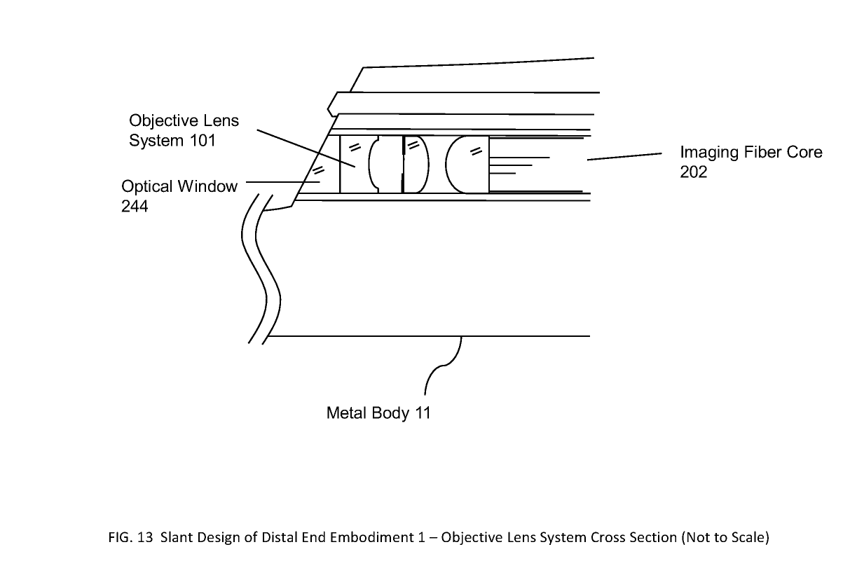

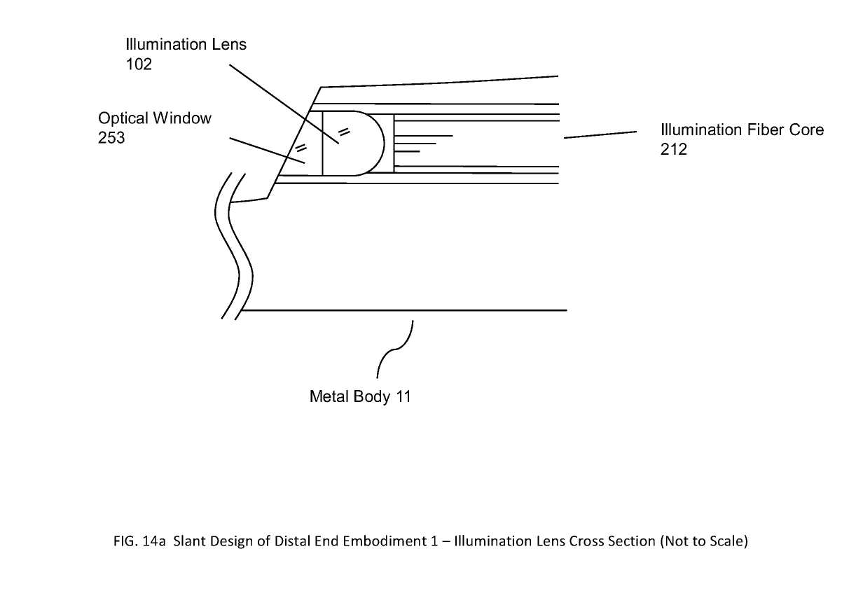

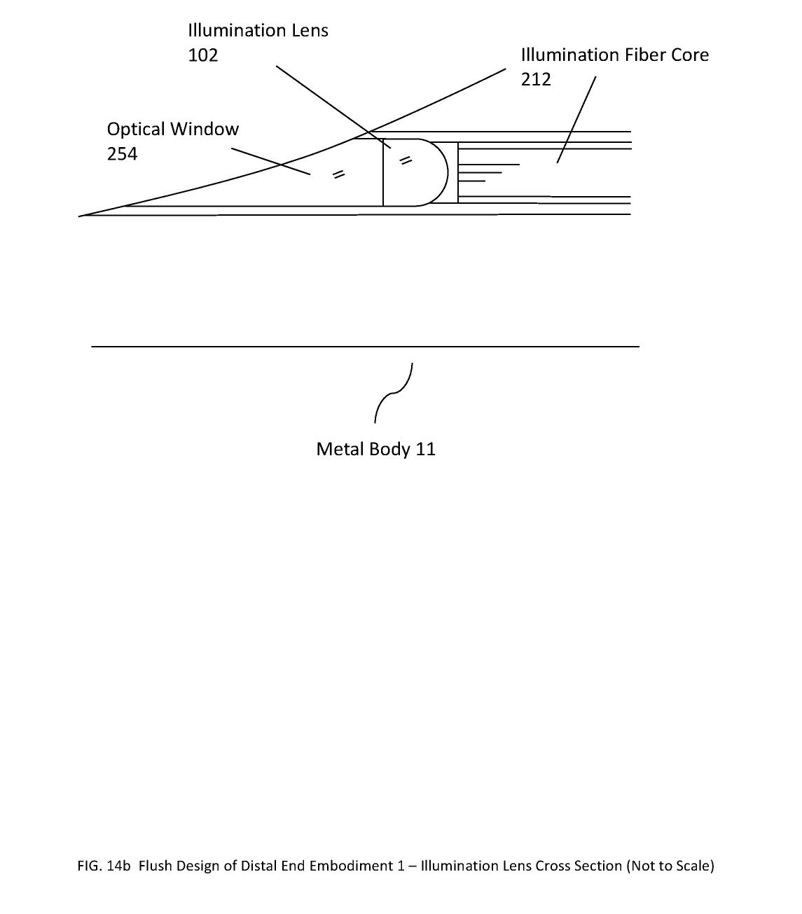

In other embodiments, the optical distal end designs in FIGS. 2 through 5 can be modified to produce slant end designs. For example, FIG. 13 shows a side view on the plane perpendicular to the IR Endoscopic Probe body 11 through the objective lens system 101, regarding the modification to the optical distal end design of FIG. 2 wherein the front is inclined at an angle theta, typically less than 40 degrees. A slant optical window 244 faces the object side of the objective lens system. FIG. 14a also shows a side view on the plane perpendicular to the IR Endoscopic Probe body 11 through one of the illumination lenses 102. A slant optical window 253 precedes the illumination lens.

In further embodiments, the distal end designs in FIGS. 2 through 5 can be modified to produce optical distal ends that are flush to the tapered tip for the objective lens system and the illumination lens systems. For example, FIG. 14b shows a side view on the plane perpendicular to the IR Endoscopic Probe body 11 through the illumination lens system 102, regarding the modification to the optical distal end design of FIG. 2. A flush optical window 254 precedes the illumination lens.

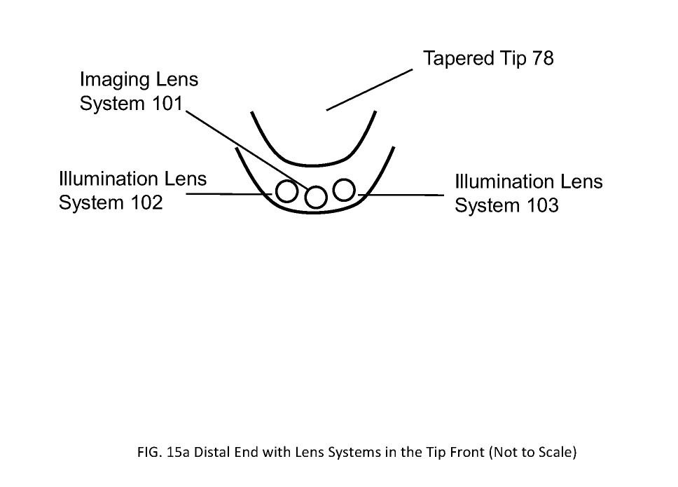

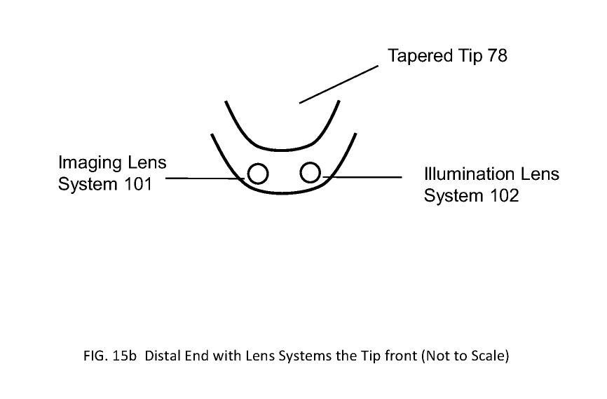

Other embodiments of the optical distal end consist of placing the objective lens system 101 and the illumination lens systems 102 and 103 in front of the tapered tip 78 for Embodiment 3 of the device as shown in FIG. 15a and FIG. 15b. In FIG. 15a as seen from the front, the objective lens system 101 is in the left vicinity of the center of the tapered tip 78 while the illumination lens system 102 is on the left side of the tapered tip 78 and the illumination lens system 103 is on the right side of the tapered tip 78. In FIG. 15b as seen from the front, the objective lens system 101 is on the left side of the tapered tip 78 while there is only one illumination lens system 102 on the right side of the tapered tip 78. In all cases, the lens systems are preceded by optical windows that are flush to the shape of the tip. An irrigation and suction conduit may be added on top of objective lens system if these designs are used with Embodiment 1 of the IR Endoscopic Probe.

The objective lens system is such that objects can be focused from 1 mm to ˜10 cm. Also, the illuminating lens system provides uniform illumination for the imaging field of view. On the other end, the fiber optics bundles provide the interface with the imaging camera optical assembly and the illumination source optical assembly. This is shown in FIG. 1.

The number of fibers in the imaging fiber core is on the order of 10,000 fibers, a trade-off number that provides excellent resolution of the object image. The imaging fiber core continues to the imaging fiber bundle in the integrated housing 14/34/69 external to the IR Endoscopic Probe handle 12/32/62. The imaging fiber core and the imaging fiber bundle are the same. They are named differently to distinguish their location in the geometry and arrangement of the IR Endoscopic Probe. The camera provides an electrical signal to a monitor to provide video to medical personnel.

The illumination source assembly illuminates the incision by transmitting light through the illumination fiber bundles and the illumination fiber cores. The illumination fiber cores and the illumination fiber bundles are the same. They are named differently to distinguish their location in the geometry and arrangement of the IR Endoscopic Probe. The illumination fiber cores and fiber bundles consist of a plurality of fibers, on the order of 300 to 1000 glass fibers each. The illumination fiber cores and bundles provide the means to carry light from the illumination source with enough intensity and low attenuation such that the emitted light at the output of the illumination lens allows the objective lens system to discern objects with clarity. The placement of the illumination fiber cores with respect to optical distal end lens is such that essentially all light can be output at the optical distal end. Another option is to implement the fiber cores with plastic fibers with a diameter of around 500 microns in which case each fiber core would consist of a single plastic fiber.

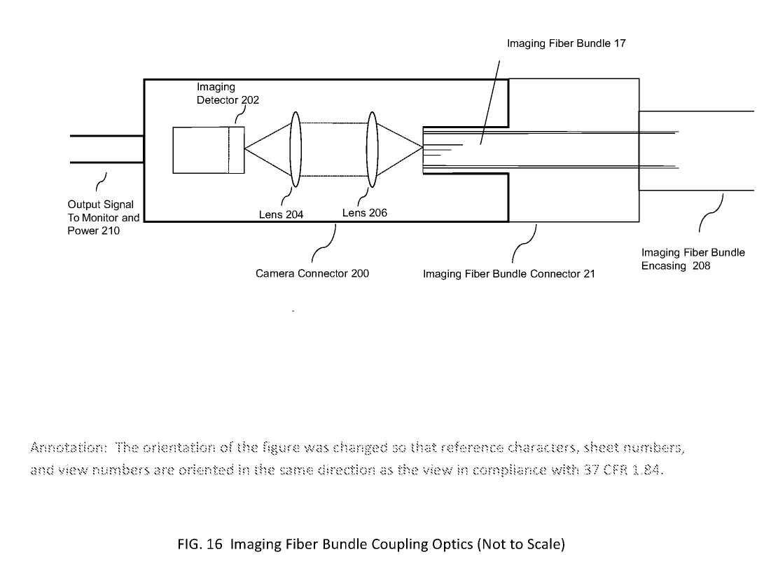

The image fiber bundle proximal end 17 terminates on a plane perpendicular to the axis of the fiber bundle as shown in FIG. 16. When the proximal end connector 21 connects to the camera through the camera connector 200, the fiber bundle output is focused by the camera lenses 204/206 so that the image is placed on the solid state imaging detector 202 plane. Light is emitted from the fiber as a function of the numerical aperture of the fiber bundle with the camera lens system performing all the focusing of the image signal onto the imaging plane. The electrical signal is sent to the video monitor through the electrical interface cable 210 which also provides power to the imaging detector 202 and associated electronics.

The proximal end of each illumination fiber bundle 40 and 41 terminates flush on a plane perpendicular to the fiber longitudinal axis as shown in FIG. 17. The illumination fiber bundle connector 22 mates with the illumination source connector 300 such that each fiber bundle is placed at a fixed distance from the illumination source lenses 304/306/308/310, which consists of a single fixture coupled to both illumination fiber bundles. The illumination source 302 radiates light in the infrared spectrum and can consist of an infrared diode or laser, for example. The illumination source lenses implement all the focusing of the infrared light into the two fiber bundles such that the imaging fiber bundles 40 and 41 capture the light as a function of their numerical aperture. One pair of lenses couple light to each illumination fiber bundle. For example, lenses 308 and 310 focus light into imaging fiber bundle 40. Each illumination source lens pair focuses light into each illumination fiber bundle so that a large fraction of the light is coupled into each illumination fiber bundle. Power is provided to the illumination source connector 300 through the electrical cable 312.

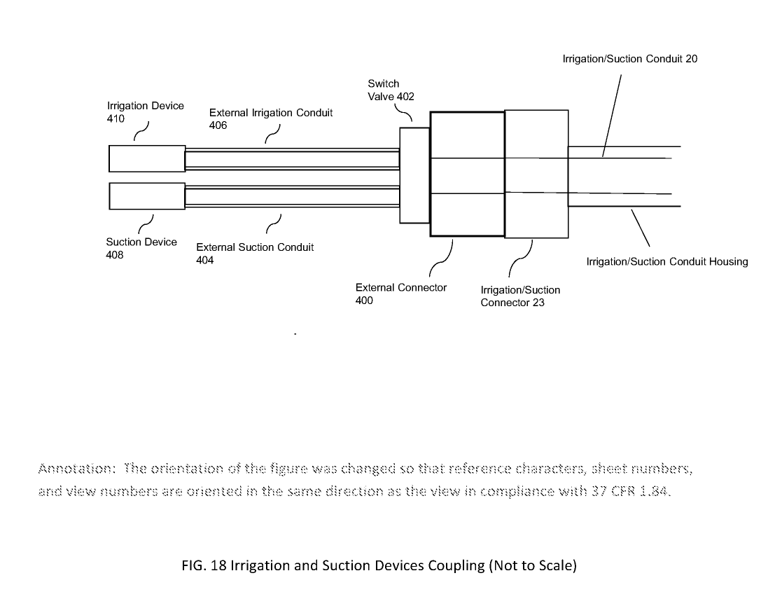

The other external interface is to an irrigation device and to a suction device. The interfaces to these devices are implemented in embodiments 1 and 2 of the IR Endoscopic Probe in FIGS. 1 and 7. FIG. 18 shows the detail of this interface. The IR Endoscopic Probe connector 23 couples to the external connector 400 while the irrigation/suction conduit 20 provides the flow to either the Irrigation Device 410 or the Suction Device 408 which are selected by the switch valve 402. The Irrigation Device 410 is coupled to the switch valve through an external irrigation conduit 406 while the Suction Device 408 couples to the switch valve 402 through the external suction conduit 404.

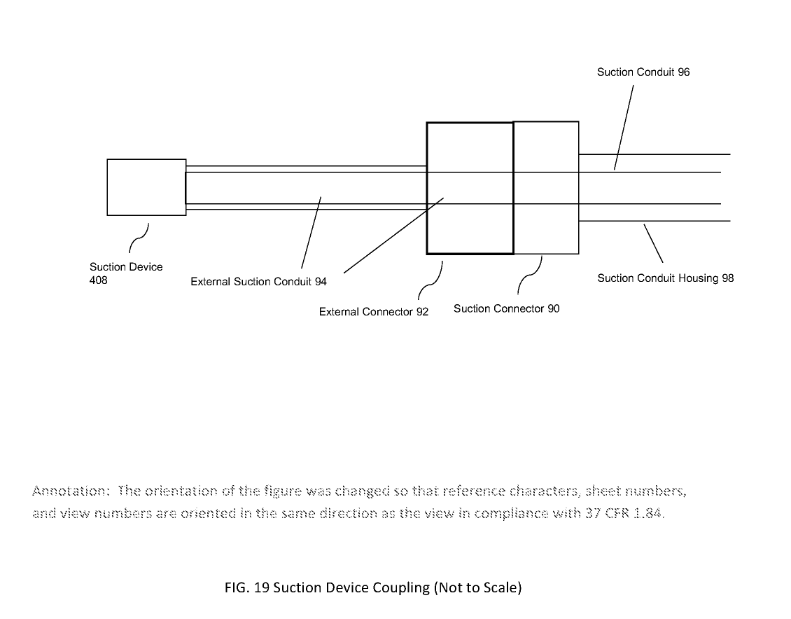

For Embodiment 2, the interface to the suction device is shown in FIG. 19. The IR Endoscopic Probe connector 90 couples to the external connector 92 while the suction conduit 96 provides the flow to either the Suction Device 408. The Suction Device 408 couples to external suction connector 90 the external conduit 94.

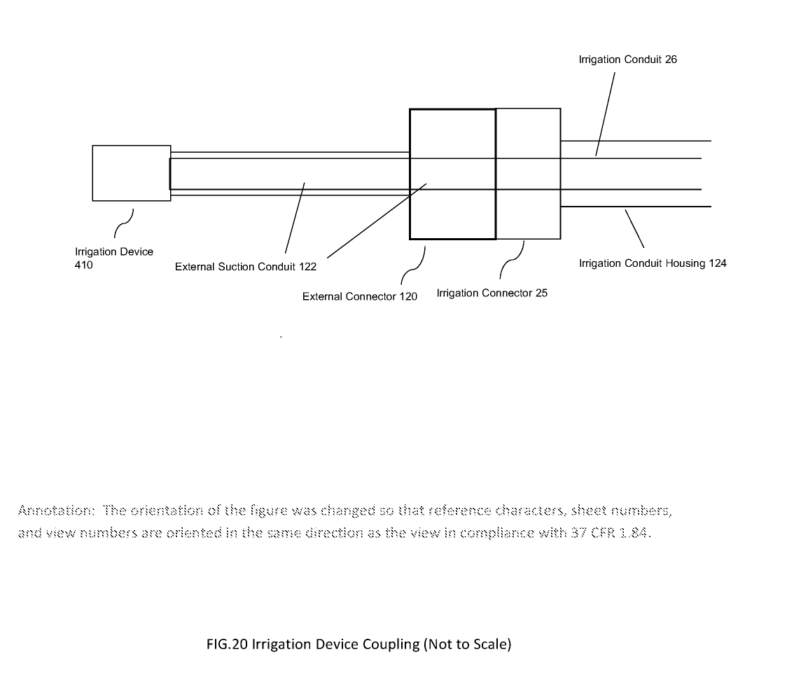

For Embodiment 2, the interface to the irrigation device is shown in FIG. 20. The IR Endoscopic Probe connector 25 couples to the external connector 120 while the irrigation conduit 26 provides the flow to the Irrigation Device 410. The Irrigation Device 410 couples to external suction connector 120 the external conduit 122.

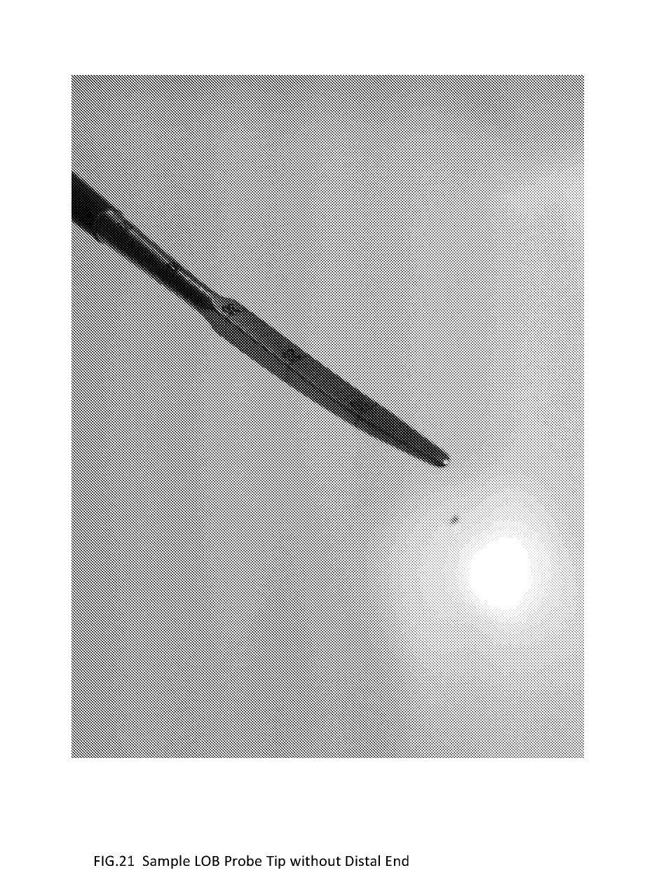

FIG. 21 shows the tapered tip of a IR Endoscopic Probe with a stainless steel fabrication. The IR Endoscopic Probe tapered tip described herein is similar in design except that the optical distal end is placed near the tip, producing a flush transition or a small step in the integrated design, or in front.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

1. An endoscopic surgical device used to create pilot holes in the pedicle bone of vertebrae for subsequent insertion of pedicle screws, to image the pedicle bone at selected infrared light wavelengths to select an optimal boring of the pedicle bone and to predict breaches of the pedicle bone outer surface to avoid rupturing of the outer nerve and vascular structures, comprising:
a tapered tip constructed of a rigid, stiff and curved metal structure to allow for a boring of the pilot hole that follows the curvature of the pedicle bone that results in sharp and smooth walls for eventual placement of pedicle screws;
an elongated metal body having a distal end and a proximal end and whose distal end is formed contiguously to said tapered tip proximal end;
an endoscope terminated in an optical distal end and integrated inside said elongated metal body through internal channels that run parallel along said elongated metal body, wherein said optical distal end is placed at a predetermined distance from said tapered tip distal end;
a handle placed in the proximal end of said elongated metal body to drive said surgical device into vertebra pedicle bone;
a fiber optics harness;
an imaging device;
and an illumination device;
wherein the tapered tip is comprised of a predetermined width at its proximal end that gradually decreases to a pointy and sharp distal end;
wherein the tapered tip is comprised of a predetermined height at its proximal end and gradually curves to said pointy and sharp distal end;
wherein the tapered tip is of a predetermined length as a function of the pedicle bone type;
wherein the elongated metal body is of a predetermined length to exert adequate torque during boring of the pilot hole;
wherein the handle structure is shaped in the form of a “T” where the parallel extension is of a predetermined length to exert adequate torque during boring of the pilot hole;
wherein said endoscope is comprised of one imaging system comprised of an imaging fiber bundle terminated in an objective lens system, two illumination systems each one comprised of an illumination fiber bundle terminated in an illumination lens system, and an irrigation and suction conduit;
wherein the optical distal end is placed on top of the tapered tip such that the field of view of the objective lens system captures the front section of the tapered tip distal end to use it as a guide during the boring of the pilot hole for imaging of the upper hemisphere mostly;
wherein the optical distal end is placed alternately in front of the tapered tip such that the field of view of the objective lens system captures the front surroundings entirely to determine the path of the pilot hole operation and the terminal point of the boring operation located in the vertebral bone;
wherein said fiber optics harness is comprised of an imaging fiber bundle encased in a plastic housing, two illumination fiber bundles encased in a plastic housing, and an irrigation and suction conduit wherein the imaging fiber bundle merges with the two illumination fiber bundles at a plastic-molded junction to emerge in a fiber optics assembly that encases individually the imaging fiber bundle and the two illumination fiber bundles in a synthetic material and wherein the irrigation and suction conduit further merges with the fiber optics assembly at a plastic-molded junction to emerge in an integrated housing that encases individually the imaging fiber bundle, the two illumination fiber bundles and the irrigation and suction conduit in a synthetic material and enters the handle;
wherein said fiber optics harness imaging fiber bundle transitions to said endoscope imaging system as the same imaging fiber bundle to provide imaging functions by connecting at its proximal end to said imaging device through one connector, wherein the two fiber optics harness illumination fiber bundles further transition to the two endoscope illumination systems individually as the same illumination fiber bundles to provide illumination functions by connecting at its proximal end to said illumination device through one connector, and wherein said fiber optics harness irrigation and suction conduit further transitions to said endoscope irrigation and suction conduit to provide irrigation and suction functions by connecting at its proximal end to irrigation and suction devices through one connector;
wherein the fiber optics harness is comprised of flexible fiber optics bundles, a flexible irrigation/suction conduit and flexible synthetic encasings of the same to allow for unobstructed manipulation of the endoscopic surgical device with the handle;
wherein the imaging device consists of a solid state imaging detector and a two-lens system to focus the image received from the fiber optics harness imaging fiber bundle onto said solid state imaging detector where the imaging device is encased in a housing that connects to said imaging fiber bundle connector;
wherein the illumination device consists of an infrared illumination source with two two-lens systems to focus the infrared light into the two illumination fiber bundles where each two-lens system is allocated to each illumination fiber bundle where the illumination device is encased a housing that connects to the two illumination fiber bundles connector;
wherein said optical distal end consists of a small vertical step less than 2 mm in height on top of said tapered tip and wherein said optical distal end is located at a predetermined distance from said tapered tip distal end;
wherein said optical distal end alternately consists of a small slant step inclined at a predetermined angle on top of said tapered tip and wherein said optical distal end is located at a predetermined distance from said tapered tip distal end wherein the objective lens system is preceded by a slant optical window inclined at said predetermined angle and wherein each illumination lens systems is preceded by a slant optical window inclined at said predetermined angle;
wherein said optical distal end alternately terminates flush on top of said tapered tip and wherein said optical distal end is located at a predetermined distance from said tapered tip distal end wherein said objective lens system is preceded by a flush optical window and wherein each illumination lens system is preceded by a flush optical window;
and wherein said optical distal end is arranged with said objective lens system in the center, one illumination lens system on left side of the objective lens system as viewed from the front, the other illumination lens system on the right side of the objective lens system as viewed from the front, and said irrigation and suction conduit on top of the objective lens system.

2. (canceled)

3. An endoscopic surgical device used to create pilot holes in the pedicle bone of vertebrae for subsequent insertion of pedicle screws, to image the pedicle bone at selected infrared light wavelengths to select an optimal boring of the pedicle bone and to predict breaches of the pedicle bone outer surface to avoid rupturing the outer nerve and vascular structures, comprising:
A tapered tip constructed of a rigid, stiff and curved metal structure to allow for a boring of the pilot hole that follows the curvature of the pedicle bone that results in sharp and smooth walls for eventual placement of pedicle screws;
an elongated metal body having a distal end and a proximal end and whose distal end is formed contiguous to said tapered tip proximal end;
an endoscope terminated in an optical distal end and integrated inside said elongated metal body through internal channels that run parallel along said elongated metal body, wherein said optical distal end is placed at a predetermined distance from said tapered tip distal end, wherein said optical distal end is alternately placed in the front of said tapered tip distal end;
a handle placed in the proximal end of said elongated metal body to drive the said surgical device into vertebra pedicle bone;
a fiber optics harness;
an imaging device;
and an illumination device;
wherein the tapered tip is comprised of a predetermined width at its proximal end that gradually decreases to a pointy and sharp distal end;
wherein the tapered tip is comprised of a predetermined height at its proximal end and gradually curves to said pointy and sharp distal end;
wherein the tapered tip is of a predetermined length as a function of the pedicle bone type;
wherein the elongated metal body is of a predetermined length to exert adequate torque during boring of the pilot hole;
wherein the handle structure is shaped in the form of a “T” where the parallel extension is of a predetermined length to exert adequate torque during boring of the pilot hole;
wherein said endoscope is comprised of one imaging system comprised of an imaging fiber bundle terminated in an objective lens system and two illumination systems each one comprised of an illumination fiber bundle terminated in an illumination lens system;
wherein the optical distal end is placed on top of the tapered tip such that the field of view of the objective lens system captures the front section of the tapered tip distal end to use it as a guide during the boring of the pilot hole for imaging of the upper hemisphere mostly;
wherein the optical distal end is placed alternately in front of the tapered tip such that the field of view of the objective lens system captures the front surroundings entirely to determine the path of the pilot hole operation and the terminal point of the boring operation located in the vertebral bone;
wherein said fiber optics harness is comprised of an imaging fiber bundle and two illumination fiber bundles wherein the imaging fiber bundle merges with the two illumination fiber bundles at a plastic-molded junction to emerge in an integrated housing that encases individually the imaging fiber bundle and the two illumination fiber bundles and enters the handle;
wherein said fiber optics harness imaging fiber bundle transitions to said endoscope imaging system as the same imaging fiber bundle to provide imaging functions by connecting at its proximal end to said imaging device through one connector, and wherein the two fiber optics harness illumination fiber bundles further transitions to the two endoscope illumination systems individually as the same illumination fiber bundles to provide illumination functions by connecting at its proximal end to said illumination device through one connector;
wherein the fiber optics harness is comprised of flexible fiber optics bundles and flexible synthetic encasings of the same to allow for unobstructed manipulation of the endoscopic surgical device with the handle;
wherein the imaging device consists of a solid state imaging detector and a two-lens system to focus the image received from the fiber optics harness imaging fiber bundle onto said solid state imaging detector where the imaging device is encased in a housing that connects to said imaging fiber bundle connector;
wherein the illumination device consists of an infrared illumination source with two two-lens systems to focus the infrared light into the two illumination fiber bundles where each two-lens system is allocated to each illumination fiber bundle where the illumination device is encased a housing that connects to the two illumination fiber bundles connector;
wherein said optical distal end consists of a small vertical step less than 2 mm in height on top of said tapered tip and wherein said optical distal end is located at a predetermined distance from said tapered tip distal end;
wherein said optical distal end alternately consists of a small slant step inclined at a predetermined angle on top of said tapered tip and wherein said optical distal end is located at a predetermined distance from said tapered tip distal end wherein said objective lens system is preceded by a slant optical window inclined at said predetermined angle and wherein each illumination lens systems is preceded by a slant optical window inclined at said predetermined angle;
wherein said optical distal end alternately terminates flush on top of said tapered tip and wherein said optical distal end is located at a predetermined distance from said tapered tip distal end wherein said objective lens system is preceded by a flush optical window and wherein each illumination lens system is preceded by a flush optical window;
wherein said optical distal end alternately terminates in the front of said tapered tip, wherein said objective lens system is preceded by an optical window geometrically flush to said front of said tapered tip and wherein said objective lens system is located on the left side of the center of said tapered tip as viewed from said front, wherein one illumination lens system is preceded by an optical window geometrically flush to said front of said tapered tip and is located on the left side of said objective lens system as viewed from said front, and wherein the other illumination lens system is preceded by an optical window geometrically flush to said front of said tapered tip and is located on the right side of the center of said tapered tip as viewed from said front;
wherein the endoscope is comprised alternately of one imaging system terminated in an objective lens system and one illumination system terminated in an illumination lens system;
wherein said fiber optics harness is comprised alternately of one imaging system and one illumination system;
wherein alternately said fiber optics harness imaging system transitions to said endoscope imaging system as the same imaging fiber bundle to provide imaging functions by connecting at its proximal end to an imaging device through one connector, and wherein said fiber optics harness illumination system further transitions to said endoscope illumination system as the same illumination fiber bundles to provide illumination functions by connecting at its proximal end to an illumination device through one connector;
wherein said optical distal end alternately terminates in the front of said tapered tip, wherein said objective lens system is preceded by an optical window geometrically flush to said front of said tapered tip and is located on the left side of the center of said tapered tip as viewed from said front and wherein said illumination lens system is preceded by an optical window geometrically flush to said front of said tapered tip and is located on the right side of the center of said tapered tip as viewed from said front;
and wherein said optical distal end is arranged alternately with the objective lens system in the center, one illumination lens system on the left side of said objective lens system as viewed from the front, and the other illumination lens system on the right side of said objective lens system as viewed from the front.

4. A method for creating pilot holes in the pedicle bone of vertebrae for subsequent insertion of pedicle screws and for imaging the pedicle bone at selected infrared light wavelengths to select an optimal boring of the pedicle bone and to predict breaches of the pedicle bone outer surface to avoid rupturing the outer nerve and vascular structures by using an endoscopic surgical device with a tapered tip in its proximal end and viewing the operation in a monitor comprising the steps of:
connecting said endoscopic surgical device to an infrared imaging device, an infrared illumination device and irrigation and suction devices to enable the endoscopic functions of said surgical device at the selected infrared light wavelength;
placing said tapered tip on the selected vertebra's pedicle at predetermined boney landmarks;
driving the tapered tip of said endoscopic surgical device a short distance into the pedicle boney tissue by manipulating said surgical device with its handle;
determining in the monitor a clear path through pedicle boney tissue away from nerve and vascular structures and the pedicle boney wall;
driving said tapered tip further into said clear path through pedicle boney tissue;
suctioning blood as needed throughout the surgical operation and flushing the optical distal end of the endoscope as needed to clear obstructions throughout the surgical operation;
continuing boring while continually ascertaining that said tapered tip is in said clear path through pedicle boney tissue away from nerve and vascular structures and the pedicle boney wall by viewing the operation in the monitor;
withdrawing said tapered tip a short distance if said tapered tip gets too close to nerve or vascular structures or the pedicle boney wall or if said tapered tip has breached the nerve or vascular structures or the pedicle boney wall by viewing the operation in the monitor;
continuing boring in a path away from nerve and vascular structures and the pedicle boney wall once said tapered tip has been withdrawn a short distance if said tapered tip has gotten too close to nerve or vascular structures or pedicle boney wall or if said tapered tip has breached the nerve or vascular structures or the pedicle boney wall;
withdrawing said tapered tip from the pedicle if the vertebral body has been reached by viewing the operation in the monitor as the pilot hole has been created.

5. (canceled)
